(19) United States
(12) Patent Application Publication
(10) Pub. No.: US 2024/0035694 A1
(43) Pub. Date: Feb. 1, 2024

(54) Method of controlling biological and environmental factors in agricultural facilities

(71) Applicants: Paul Ruiz, Las Vegas, NV (US); Travis Olson, Menomonie, WI (US); Josh Oelker, White Bear Township, MN (US)

(72) Inventors: Paul Ruiz, Las Vegas, NV (US); Travis Olson, Menomonie, WI (US); Josh Oelker, White Bear Township, MN (US)

(21) Appl. No.: 18/342,638

(22) Filed: Jun. 27, 2023

Related U.S. Application Data
(60) Provisional application No. 63/356,115, filed on Jun. 28, 2022.

(51) Int. Cl. F24F 11/00; F24F 8/10; F24F 8/22; F24F 8/30

(52) U.S. Cl. CPC F24F 11/0001; F24F 8/10; F24F 8/22; F24F 8/30

(57) ABSTRACT

A method to maximize a farm's biosecurity and reduce stressors originating from environmental factors such as heat stress for example. This approach provides the ultimate in biosecurity while concurrently controlling the environment to precise metrics. The aforementioned is done by utilizing a unique combination of several components in varying combinations to achieve the facilities best outcome through control via several systems; Supply Air System/s to control air flow and quality into the facility or room, Discharge Air System/s to control air flow and air quality exiting the facility or room, BioSecurity System/s to control the bioload within the air entering and leaving the facility or room, and the Controls System/s to manage the three aforementioned systems.

### CROSS-REFERENCE TO RELATED APPLICATIONS

This application claims the benefit and priority of Provisional Patent Application Ser. No. 63/356,115 filed Jun. 28, 2022, for a Method of controlling biological and environmental factors in agricultural facilities under 35 U.S.C. § 119(e), incorporated herein by reference in its entirety for continuity of disclosure.

### BACKGROUND OF THE INVENTION

### Field of the Invention

The field of the invention is related to a method for controlling air going in or out of a protein producing facility to maintain desired air parameters and bioloads.

### Background Information

Previous methods to control heat stress or prevent disease spread in protein production facilities have not been effective. Heat stress related illnesses and disease are the main factors contributing to production losses, and even death. These issues can result in significant financial losses for the producers. Ultimately, the industry requires a method that can concurrently manage heat stress and biosecurity.

### SUMMARY OF THE INVENTION

The applicant has identified that the current methods of reducing environmental stressors and illnesses severely limits the efficiency of the protein production industry. This method perfectly controls the animals' environment, ending heat stress. Giving protein producers optimal thermal and environmental manipulation reduces dependency on antibiotics, hormone therapy, and unnecessary feed additives. Aspects of the invention present added and unexpected benefits in the regulation of air flow, especially in an agricultural environment, this optimum breeding environment increases feed efficiency, average daily gain, fertility, and overall survival rate. Our system also decreases death loss, improves gut health, improves lifetime productivity, and improves eatability.

Vital to these improvements is the addition to reducing pathogen vectors like the Porcine Respiratory complex, Influenza, and Salmonella. Elimination of such vectors reduces dependency on antibiotics and hormone therapy. It also reduces the risk of unnecessary feed additives and makes for healthier animals while increasing yield and, ultimately, profit. This is the first and only system that successfully disinfects all airborne infectious diseases entering and leaving production facilities through use of our method.

In one aspect the invention includes a method for controlling fomite transmission of pathogens into the facility through a zoning approach to facilitate a predictable clean/dirty line within a facility and/or room or externally to a facility and/or room. Each facility is designed to maximize the farm's biosecurity and reduce stressors originating from environmental factors. Managing the use of the following systems within this method provides the best in air management and disease control unparalleled by anything in the industry; Supply Air System/s, Discharge Air System/s, BioSecurity System/s, and the Controls System/s.

The above partial summary of the present invention is not intended to describe each illustrated embodiment, aspect, or every implementation of the present invention. The figures and detailed description and claims that follow more particularly exemplify these and other embodiments and further aspects of the invention.

### BRIEF DESCRIPTION OF DRAWINGS

The invention may be more completely understood in consideration of the following description of various embodiments of the invention in connection with the accompanying drawings, in which:

FIG. 1 is a top view of a biosecurity control system in accordance with one

aspect of the present invention with a top of a facility removed for clarity.

FIG. 2 is a side view of an air flow control system and a biosecurity control system in accordance with one aspect of the present invention with a side of a facility removed for clarity.

While the invention is amenable to various modifications and alternative forms, specifics thereof have been shown by way of example in the drawings and will be described in detail. It should be understood, however, that the intention is not necessarily to limit the invention to the particular embodiments, aspects and features described. On the contrary, the intention is to cover all modifications, equivalents, and alternatives falling within the spirit and scope of the invention and as defined by the appended claims.

### DETAILED DESCRIPTION OF THE INVENTION

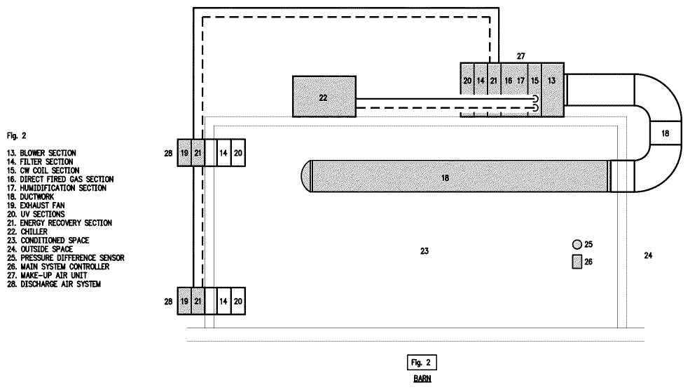

The first system to be discussed is the Supply Air System Referring to FIGS. 2,

aspects of the systems and methods are shown. This equipment 22,27 encompassing 13-22, 25,26 is the primary means of moving air into the facility by controlling the pressure of the building from but not limited to negative and/or positive pressure as needed and providing systems to manipulate the air delivered into the space/s by means including but not limited to variable and/or constant speeds, volumes, temperature, humidity/s and/or air quality metrics.

In an ideal embodiment the following components may be included in verifying quantities, capacities, or configurations. The first supply air system component is the fan 13 system to move air into the facility 23 at varying air flow rates and quantities. The second supply air system component is the air filter/s 14 to harvest particulates out of the air supplied to the barn environment 23 from the outside 24. The third supply air system component is the cooling system 15,22 to reduce latent and/or sensible loads in the supply air being supplied to a designated space 23. Heat can be removed from an airflow by routing a fluid and or gas having a lower temperature than that of the airflow within the respective heat exchanging coil 15. In some embodiments the cooling system 15,22 may also be utilized to lower the space 23 relative humidity. Each of the respective heat exchanging coils can include a heating coil and/or a cooling coil. In an embodiment a variable rate pump 22 can be used to control a flow rate of fluid routed through the respective heat exchanging coil. A variable speed fan can be used to draw the respective mixed airflow through the respective heat exchanging coil so as to control a flow rate of the respective mixed airflow.

The fourth supply air system component is the heating system 16 to increase latent and/or sensible loads. The heat can be added or removed using heat exchanging coils 16 or through direct fired heaters 16. Heat can be added to an airflow by routing a fluid and/or gas having a higher temperature than that of the airflow within the respective heat exchanging coil 16 the space 23. Each of the respective heat exchanging coils can include a heating coil and/or a cooling coil. In an embodiment A variable rate pump can be used to control a flow rate of fluid routed through the respective heat exchanging coil.

The fifth supply air system component to be discussed is the heat recovery system 21 to recover latent loads from the exhaust air system 28 in order to temper the supply air and/or reduce energy demands.

The sixth supply air system component to be discussed is the humidification system 17 meant to raise the space 23 relative humidity. In an embodiment, the humidity control device 17 has a tank for storing water provided from the water supply device, and is provided with an independent drain pipe or is treated together with an air conditioner pipe. Provide a humidity control system 17. In addition, the humidity control system 17 of the present invention can adjust to different humidity corresponding to each different humidity control environment, and the humidity sensor 26 automatically detects the humidity of the humidity control environment 23 by the humidity sensor 26. The humidity control device 17 is switched between a humidified state and a dehumidified state according to each demand, and each air-conditioned area 23 can always maintain an appropriate humidity, and sanitize the entering air from the outside 24 to be provided through the supply air system 22,27 encompassing 13-22, 25,26 the animal with its optimal environmental growing conditions within the space 23 while simultaneously eliminating biosecurity vectors through the air from entering the facility 23.

The seventh supply air system component encompasses the supply air distribution system 18 through but not limited to metal, fabric, polymer based, and or synthetic ductwork 18 either permanent and/or interchangeable to distribute the air into said space 23.

In a preferred embodiment a system utilizing the seven components equipment 22,27 encompassing 13-22, 25,26 as aforementioned would allow an animal to be housed in a facility 23 that would maintain an indoor climate at 65 degrees Fahrenheit +/−1 degree, relative humidity at 50%+/−10%, provide positive pressure inside 23 the building to reduce infiltration concerns, sanitize the incoming air from outside 24.

The second system to be discussed is the Discharge air system. Referring to FIGS. 2, aspects of the systems and methods are shown. This equipment 28 encompassing 14,19-21, The discharge air system 28 is used as the primary means of exhausting air from the facility 23 through passive or mechanical means to the outside 24. This system 28 aids in maintaining the desired building 23 pressure as well as increasing biosecurity, improving energy efficiency, and scrubbing the discharged air of unwanted contaminants to the outside 24. The aforementioned equipment 28 encompassing 14,19-21 may be included but is not limited to damper/s, louver/s, fan/s 19, Filter/s 14, Ionization, ultraviolet disinfection 20, and energy recovery 21 and/or combinations of the aforementioned components. In the ideal embodiment, the discharge air system 28 provides a means for exhausting disinfected air from the space 23 external to the controlled indoor environments through the use of filtration and ultraviolet disinfection to the outside 24.

The third system to be discussed is the Biosecurity system. Referring to FIGS. 2, aspects of the systems and methods are shown. This system encompasses 14,20 The biosecurity components involved within our systems are utilized in several variations to control multiple vectors. We utilize but are not limited to ultraviolet disinfection 20, filtration 14, ionization, and/or oxidation for both air and surface disinfection applications. In some embodiments the supply air 27 is filtered 14 to reduce particulate and bioloads to a percentage as determined by the user and then treated with ultraviolet disinfection 20 to disinfect the air and bioloads to a percentage as determined by the user. We also apply technologies in but not limited to systems discussed with the air systems 27,28 and/or the space 23 that utilize varying ionization and oxidation approaches to reduce particulate bioloads. These components may and/or may not be used in the supply and/or discharge air depending on site requirements.

In some embodiments the discharge air 28 is filtered 14 to reduce particulate, volatile organic compound contaminant levels, and bioloads to a percentage as determined by the user and then treated with ultraviolet disinfection 20 to disinfect the air leaving the indoor environment 23 to a percentage as determined by the user. Ionization is used to reduce volatile organic compounds and/or particulate loads in and/or discharged by the exhaust air system. Each system is uniquely designed to maximize each of the components effectiveness based on the requirements of each space where these systems may or may not be applied in varying capacities and quantities.

In some embodiments a ultraviolet disinfection system 20 and filtration 14 may/or may not be utilized and is not limited to varying capacities, quantities, and configurations to provide a clean room system to reduce and or eliminate airborne and/or fomite vectors from being transmitted into and/or out of one and/or multiple spaces adjoining or directly connected to one and/or multiple spaces within the spaces incorporating but not limited to varying capacities, quantities, and or configurations of the biosecurity system/s, supply air system/s, discharge air system/s, and/or control system/s.

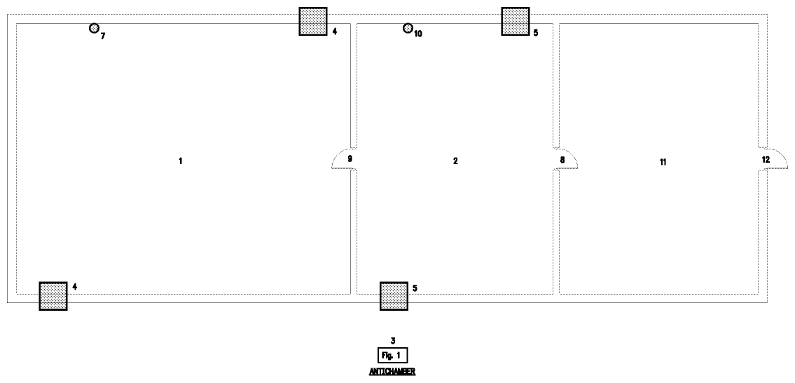

Referring to FIGS. 1, aspects of the Clean Room system/s and method/s are shown In an ideal embodiment, a biohazard anteroom is utilized to prevent dangerous substance from leaking outside and/or inside the handling chamber(conditioned space) thereof and to prevent the handling chamber from being polluted with dust, etc., introduced from the outside by keeping the chamber pressure difference of both an anteroom and the dangerous substance handling room constant.

The airtight doors 9, 8 which are not simultaneously opened and closed are provided to the respective entrances of both the outside 3 and a dangerous substance handling chamber 1 from a biohazard anteroom 2 and two sets 4, 5 of fan units equipped with particulate removal and ultraviolet disinfection sterilization are provided for room 2 and the chamber 1 in order to make the inside pressure of the room 2 lower than the chamber 1. A pressure difference sensor 7,10 for detecting the pressure difference of the room 2 and the chamber 1 is provided and the pressure difference of the room 2 and the chamber 1 is held constant by controlling the actuation of the fan units 4, 5. As a result, the dangerous substance is prevented from leaking outside chamber 1 and also chamber 1 is not polluted with dust, etc., introduced from the outside 3.

A clothing change room 11 may be included in the passageway before the two self-closing doors 8,12 of room 11 to further prevent a targeted dangerous substance from leaking outside and/or inside the handling chamber 1(conditioned space) and/or anteroom 2.

The fourth system to be discussed is the controls system. Referring to FIGS. 2, aspects of the systems and methods are shown. The control system 26 is customized to meet the scale of the users needs and/or systems needs as designed for optimal effectiveness. The method and quantity of control points needed by which each system and/or systems utilized in or accompanied with but not limited to the supply air 22,27, discharge air 28, and biosecurity systems 14,20 is controlled and/or utilized to provide functionality to the three aforementioned systems is customizable to a single and/or multiple control point/s up to limiting factors for the control system 26 at any given time. In some embodiments the user is able to monitor several indoor air quality metrics in several buildings or several spaces within one building. In other embodiments, the control system has the capability to be utilized but not limited to local control and/or monitoring by users on site through an onsite digital platform and/or other means of control by remote monitoring and/or control.

The control system 26 is able to monitor and/or control the supply air system 28 and/or discharge air system 28 to maintain building pressure. In some embodiments a positive pressure is required in the space, therefore the control system monitors the building pressure through one or in other embodiments several pressure sensors 25 to slow the exhaust rate of the exhaust system 28 to provide back pressure in the space 23. This provides a positive pressure in the space 23 in relation to the outside 24 environment as the supply air flow rate is maintained. In other embodiments the control system 26 maintains the supply air system 27 air flow rate and provides air flow rate through the exhaust air system 28 in excess of the supply air system 27 this allows the user to control the space 23 in a negative pressure in relation to the outside 24 environment. The control system 26 is capable of manipulating the supply air and discharge air at varying rates and flows to achieve specific airflow requirements to meet the needs of the user. Some requirements for varying airflow rates through the space 23 are but are not limited to stocking density, heating demand, cooling demand, air quality requirements, or/and or biosecurity requirements as defined by the user.

In some embodiments the control system 26 may but is not limited to controlling the cooling temperature to 65 degrees fahrenheit+or−1 degree fahrenheit. and heating to 70 degrees fahrenheit+or−1 degree fahrenheit, humidity at 50 percent +/−1 percent, and airflow at 6,000 Cubic Feet Per Minute +/−1 Cubic Feet Per Minute supplied to the space 23.

The terms and descriptions used herein are set forth by way of illustration only and are not meant as limitations. Those skilled in the art will recognize that many variations are possible within the spirit and scope of the invention as defined in the following claims, and their equivalents, in which all terms are to be understood in their broadest possible sense unless otherwise specifically indicated. While the particular METHOD OF CONTROLLING BIOLOGICAL AND ENVIRONMENTAL FACTORS IN AGRICULTURAL FACILITIES herein shown and described in detail is fully capable of attaining the above-described aspects of the invention, it is to be understood that it is the presently preferred embodiment of the present invention and thus, is representative of the subject matter which is broadly contemplated by the present invention, that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” Moreover, it is not necessary for a device or method to address each and every problem sought to be solved by the present invention, for it to be encompassed by the present claims.

1. A method for reducing heat stress and increasing biosecurity in the protein production industry, comprising:
The group consisting of the means to control supply air of a designated space, the means to control discharge air of a designated space, the means to control biosecurity entering a designated space, and the means to control biosecurity leaving a designated space or a mixture thereof.

2. The method of claim 1, comprising the group consisting of mechanical or natural ventilation or a mixture thereof to manage supply air.

3. The method of claim 1, further comprising the group consisting of mechanical or natural ventilation or a mixture thereof to manage discharge air.

4. The method of claim 2, further comprising the means to control latent and sensible capacity within the air-stream.

5. The method of claim 2, wherein a biosecurity system is used for air entering the facility and is controlled by the group consisting of ultraviolet disinfection, filtration, ionization, and oxidation or a mixture thereof.

6. The method of claim 3, wherein a biosecurity system is used for air exiting the facility and is controlled by the group consisting of ultraviolet disinfection, filtration, ionization, and oxidation or a mixture thereof.
